(19) United States
(12) Patent Application Publication
(10) Pub. No.: US 2026/0347298 A1
(43) Pub. Date: Nov. 5, 2026

(54) METHOD FOR PRODUCING BINAPHTHYL CARBOXYLIC ACID

(71) Applicant: HONSHU CHEMICAL INDUSTRY CO., LTD., Chuo-ku, Tokyo (JP)

(72) Inventor: Masato KODERA, Wakayama-shi (JP)

(21) Appl. No.: 19/119,686

(22) PCT Filed: Nov. 1, 2023
(86) PCT No.: PCT/JP2023/039371

§ 371 Date: Apr. 9, 2025

(30) Foreign Application Priority Data
Nov. 18, 2022 (JP) 2022-184808

(51) Int. Cl. C07C 51/367

(52) U.S. Cl. CPC C07C 51/367

(57) ABSTRACT

An object is to provide a method for producing a binaphthyl carboxylic acid having a good hue and suitable as a resin raw material having excellent optical properties. As a solution, a method for producing a 2,2′-bis(carboxymethoxy)-1,1′-binaphthyl compound represented by formula (2), including performing steps (i) to (iii) below in sequence using a 1,1′-binaphthyl-2,2′-diol compound represented by formula (1) as a starting material, is provided.
(i): A step of subjecting the 1,1′-binaphthyl-2,2′-diol compound represented by formula (1) and a halogenated acetic acid ester compound represented by formula (3) to an etherification reaction under an atmosphere or stream with an oxygen concentration of 6 vol % or less to produce a 2,2′-bis(alkoxycarbonylmethoxy)-1,1′-binaphthyl compound represented by formula (4).
(ii): A step of hydrolyzing the 2,2′-bis(alkoxycarbonylmethoxy)-1,1′-binaphthyl compound represented by formula (4) and then performing an acid treatment.
(iii): A step of performing crystallization to obtain the 2,2′-bis(carboxymethoxy)-1,1′-binaphthyl compound represented by formula (2).

### TECHNICAL FIELD

The present invention relates to a method for producing a binaphthyl carboxylic acid. The invention particularly relates to a production method for obtaining a 2,2′-bis(carboxymethoxy)-1,1′-binaphthyl compound having a good hue.

### BACKGROUND ART

Polyester resins and polyester carbonate resins produced using binaphthyl carboxylic acid components as polymerization components have excellent optical properties such as high refractive indices and low birefringence and have high levels of heat resistance, and thus have recently been expected to be materials for optical members such as optical disks, transparent conductive substrates, and optical filters. In particular, resins produced using 2,2′-bis(carboxymethoxy)-1,1′-binaphthyl (hereinafter referred to as “compound A”) represented by the following chemical formula as a polymerization component have been attracting attention for their particularly excellent optical properties (see, for example, PTLs 1 to 4).

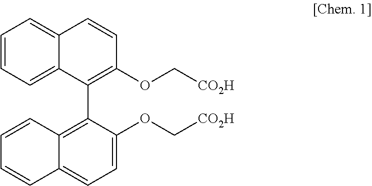 [SMILES: O=C(O)COc1ccc2ccccc2c1-c1c(OCC(=O)O)ccc2ccccc12]

One known method for producing compound A represented by the above formula is to react 1,1′-binaphthalene-2,2′-diol with a halogenated acetic acid ester such as ethyl chloroacetate and hydrolyze the resulting diester, as shown by a reaction formula below (see, for example, PTL 5). However, compound A obtained by this reaction is often used in the form of an acid chloride compound converted from an unpurified crude product with thionyl chloride, oxalyl chloride, or the like.

For materials for optical members, not only their refractive index and transmittance but also their hue may be a challenge, and coloring hinders application to optical members. However, no reports have yet been made on a method for producing a binaphthyl carboxylic acid having a good hue.

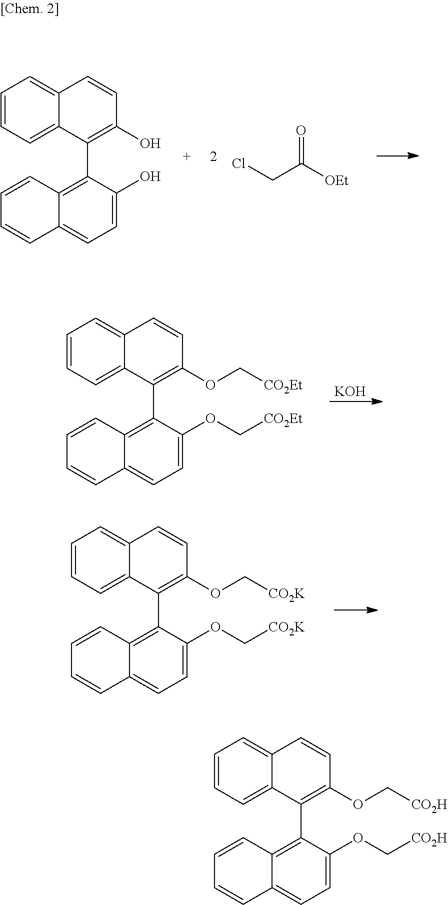

### CITATION LIST

### Patent Literature

PTL 1: Japanese Unexamined Patent Application Publication No. 2001-072872
PTL 2: Japanese Unexamined Patent Application Publication No. 2018-002893
PTL 3: Japanese Unexamined Patent Application Publication No. 2018-002894
PTL 4: Japanese Unexamined Patent Application Publication No. 2018-002895
PTL 5: Japanese Unexamined Patent Application Publication No. 2008-024650

### SUMMARY OF INVENTION

### Technical Problem

The present invention has been made in view of the foregoing circumstances, and an object thereof is to provide a method for producing a binaphthyl carboxylic acid having a good hue and suitable as a resin raw material having excellent optical properties.

### Solution to Problem

To achieve the above object, the present inventors have conducted intensive studies and found that an etherification reaction under an atmosphere or stream with an oxygen concentration of 6 vol % or less provides a binaphthyl carboxylic acid having a good hue, thereby completing the present invention.

The present invention is as follows.
1. A method for producing a 2,2′-bis(carboxymethoxy)-1,1′-binaphthyl compound represented by formula (2), including performing steps (i) to (iii) below in sequence using a 1,1′-binaphthyl-2,2′-diol compound represented by formula (1) as a starting material.
(i): A step of subjecting the 1,1′-binaphthyl-2,2′-diol compound represented by formula (1) and a halogenated acetic acid ester compound represented by formula (3) to an etherification reaction under an atmosphere or stream with an oxygen concentration of 6 vol % or less to produce a 2,2′-bis(alkoxycarbonylmethoxy)-1,1′-binaphthyl compound represented by formula (4).
(ii): A step of hydrolyzing the 2,2′-bis(alkoxycarbonylmethoxy)-1,1′-binaphthyl compound represented by formula (4) and then performing an acid treatment.
(iii): A step of performing crystallization to obtain the 2,2′-bis(carboxymethoxy)-1,1′-binaphthyl compound

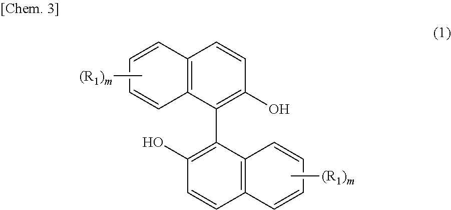

(In the formula, each R₁ independently represents a linear or branched alkyl group having 1 to 10 carbon atoms, a cyclic alkyl group having 5 to 10 carbon atoms, or an aromatic hydrocarbon group having 6 to 10 carbon atoms, and each m independently represents 0 or an integer of 1 to 4.)

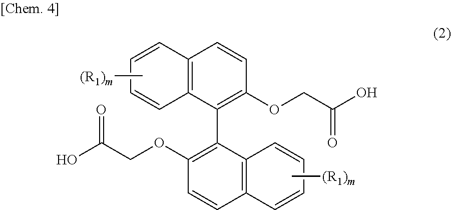

(In the formula, R₁ and m are as defined in formula (1).)

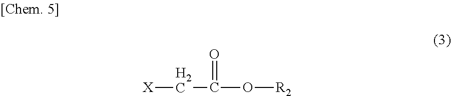

(In the formula, X represents a halogen atom, and R₂ represents a linear or branched alkyl group having 1 to 10 carbon atoms.)

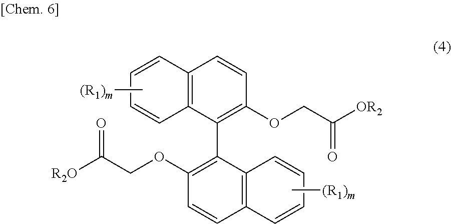

(In the formula, R₁ and m are as defined in formula (1), and each R₂ is independently as defined in formula (3).)
2. The method for producing a 2,2′-bis(carboxymethoxy)-1,1′-binaphthyl compound according to 1., wherein the crystallization in the step (iii) is performed twice.
3. The method for producing a 2,2′-bis(carboxymethoxy)-1,1′-binaphthyl compound according to 1., wherein all of the steps (ii) to (iii) are performed under an atmosphere or stream of an inert gas.

### Advantageous Effects of Invention

According to the present invention, a binaphthyl carboxylic acid having a good hue can be obtained.

The production method according to the present invention is very useful in industrial use of resin raw materials and the like.

### DESCRIPTION OF EMBODIMENTS

The present invention will now be described in detail.

In the reaction in the production method according to the present invention, as shown by the following reaction formula, a 1,1′-binaphthyl-2,2′-diol compound represented by formula (1) as a starting material and a halogenated acetic acid ester compound represented by formula (3) are subjected to an etherification reaction to produce a 2,2′-bis(alkoxycarbonylmethoxy)-1,1′-binaphthyl compound represented by formula (4), and the obtained 2,2′-bis(alkoxycarbonylmethoxy)-1,1′-binaphthyl compound is hydrolyzed and then subjected to an acid treatment to obtain a 2,2′-bis(carboxymethoxy)-1,1′-binaphthyl compound represented by formula (2).

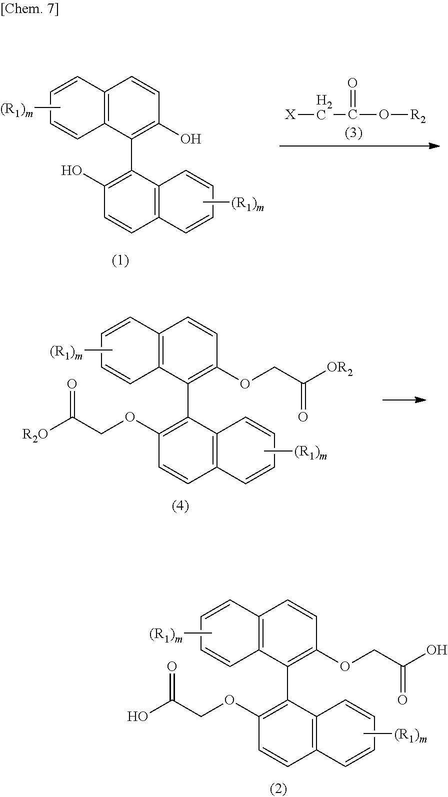

(In the formula, R₁ and m are as defined in formula (1), and each R₂ is independently as defined in formula (3).)

Each R₁ in formulas (1), (2), and (4) independently represent a linear or branched alkyl group having 1 to 10 carbon atoms, a cyclic alkyl group having 5 to 10 carbon atoms, or an aromatic hydrocarbon group having 6 to 10 carbon atoms. In particular, a linear or branched alkyl group having 1 to 6 carbon atoms or an aromatic hydrocarbon group having 6 or 10 carbon atoms is preferred. The alkyl group is preferably an alkyl group having 1 to 4 carbon atoms, more preferably an alkyl group having 1 or 2 carbon atoms. Specific examples of the alkyl group include a methyl group, an ethyl group, a n-propyl group, an isopropyl group, a n-butyl group, a t-butyl group, a n-pentyl group, a 2-methylpentyl group, and a cyclohexyl group. The aromatic hydrocarbon group having 6 to 10 carbon atoms may be substituted with an alkyl group having 1 to 4 carbon atoms, and the number of carbon atoms includes the number of carbon atoms of the substituent alkyl group. Specific examples include a 4-methylphenyl group, 4-isopropylphenyl, a phenyl group, and a naphthyl group.

In formulas (1), (2), and (4), each m independently represents 0 or an integer of 1 to 4. In particular, 0 or an integer of 1 or 2 is preferred.

X in formula (3) represents a halogen atom. Specific examples include a fluorine atom, a chlorine atom, a bromine atom, and an iodine atom, among which a chlorine atom and a bromine atom are preferred, and a chlorine atom is more preferred.

Each R₂ in formulas (3) and (4) independently represents a linear or branched alkyl group having 1 to 10 carbon atoms. In particular, a linear or branched alkyl group in which carbon bonded to an oxygen atom is primary or secondary carbon is preferred, a linear or branched alkyl group in which carbon bonded to an oxygen atom is primary carbon is more preferred, and in particular, a linear alkyl group is still more preferred. The alkyl group is preferably an alkyl group having 1 to 8 carbon atoms, more preferably an alkyl group having 1 to 4 carbon atoms, still more preferably an alkyl group having 1 or 2 carbon atoms. Specific examples include a methyl group, an ethyl group, an isopropyl group, a n-propyl group, an isobutyl group, and a n-butyl group.

Specific examples of the 2,2′-bis(carboxymethoxy)-1,1′-binaphthyl compound represented by formula (2), which is the final target, in the production method according to the present invention include 2,2′-bis(carboxymethoxy)-1,1′-binaphthyl (“compound A”), 2,2′-bis(carboxymethoxy)-6,6′-diphenyl-1,1′-binaphthalene, 2,2′-bis(carboxymethoxy)-3,3′-dimethyl-1,1′-binaphthalene, and 2,2′-bis(carboxymethoxy)-4,4′-dimethyl-1,1′-binaphthalene.

Specific examples of the halogenated acetic acid ester compound represented by formula (3) in the production method according to the present invention include methyl chloroacetate, ethyl chloroacetate, propyl chloroacetate, methyl bromoacetate, ethyl bromoacetate, propyl bromoacetate, methyl iodoacetate, ethyl iodoacetate, and propyl iodoacetate. Two or more halogenated acetic acid ester compounds whose R₂ moieties in formula (3) are different may be used in combination, but for the step (ii) to be simple and easy, it is preferable to use a single halogenated acetic acid ester compound.

<Step (i)>

### (Oxygen Concentration)

The step (i) in the production method according to the present invention is a step of subjecting a 1,1′-binaphthyl-2,2′-diol compound represented by formula (1) and a halogenated acetic acid ester compound represented by formula (3) to an etherification reaction under an atmosphere or stream with an oxygen concentration of 6 vol % or less to produce a 2,2′-bis(alkoxycarbonylmethoxy)-1,1′-binaphthyl compound represented by formula (4). The oxygen concentration is preferably 5 vol % or less, more preferably 4.5 vol % or less, still more preferably 4 vol % or less.

As the atmosphere or stream with an oxygen concentration of 6 vol % or less in the step (i), specifically, for example, an inert gas such as nitrogen, argon, or helium is used, or air mixed with an inert gas such as nitrogen gas so as to have an oxygen concentration of 6% or less is used. From an economic perspective, a mixture of air and an inert gas is preferred, and a mixture of air and nitrogen is more preferred. The lower limit of the oxygen concentration is preferably 1 vol % or more, more preferably 2 vol % or more.

### (Reaction Conditions)

In the step (i) in the production method according to the present invention, the halogenated acetic acid ester compound represented by formula (3) is preferably used in an amount of 2.1 to 3.0 mol, more preferably used in an amount of 2.2 to 2.8 mol, relative to 1 mol of the 1,1′-binaphthyl-2,2′-diol compound represented by formula (1).

In the step (i), the 1,1′-binaphthyl-2,2′-diol compound represented by formula (1) is preferably formed into a salt with a base before being reacted with the halogenated acetic acid ester compound represented by formula (3). The base is not particularly limited, and alkali metal carbonates such as lithium carbonate, sodium carbonate, and potassium carbonate, alkali metal hydrogen carbonates such as sodium hydrogen carbonate and potassium hydrogen carbonate, alkali metal hydroxides such as lithium hydroxide, sodium hydroxide, and potassium hydroxide, and organic bases such as triethylamine and pyridine are suitable for use. In particular, sodium carbonate and potassium carbonate are preferred. The amount of the base used is preferably 2.0 to 2.5 mol, more preferably 2.05 to 2.15 mol, relative to 1 mol of the 1,1′-binaphthyl-2,2′-diol compound represented by formula (1).

A preferred method of forming the 1,1′-binaphthyl-2,2′-diol compound represented by formula (1) into a salt with a base is as follows: the 1,1′-binaphthyl-2,2′-diol compound represented by formula (1) and the base are loaded into a reaction vessel together with a reaction solvent described below, and heated with stirring and held for about 10 minutes to 1 hour to form a salt. In this salt-forming method, it is more preferable to also load an alkali metal iodide described below into the reaction vessel to form a salt.

In the step (i), it is preferable to use a chain or cyclic ketone solvent having 5 to 8 carbon atoms or a chain nitrile solvent having 2 to 6 carbon atoms. Specific examples include diethyl ketone, methyl isobutyl ketone, methyl amyl ketone, 2-octanone, cyclopentanone, cyclohexanone, acetonitrile, and propanenitrile. Of these, methyl isobutyl ketone and acetonitrile are preferred. The amount of the solvent used is preferably in the range of 150 to 500 parts by weight, more preferably in the range of 200 to 300 parts by weight, relative to 100 parts by weight of the 1,1′-binaphthyl-2,2′-diol compound represented by formula (1).

The reaction temperature in the step (i) is preferably in the range of 50 °C to 130 °C, more preferably in the range of 70 °C to 120 °C, still more preferably in the range of 90 °C. to 110 °C. The reaction is typically carried out under normal pressure, but depending on the boiling point of a solvent used, the reaction may be carried out under increased pressure or reduced pressure so that the reaction temperature falls within the above range. However, the reaction is preferably carried out under normal pressure.

In the step (i) of the production method according to the present invention, the reaction with the halogenated acetic acid ester compound represented by formula (3) after the formation of the 1,1′-binaphthyl-2,2′-diol compound represented by formula (1) into a salt with a base is preferably carried out by a method in which the salt is formed, and then the halogenated acetic acid ester compound represented by formula (3) is added dropwise thereto with stirring at the above reaction temperature to cause the reaction, more preferably carried out by this method in which the salt is formed by the above-described method. If the dropping time is too long, the reaction time becomes longer accordingly, and if the dropping time is too short, the temperature control becomes difficult due to heat generation depending on the reaction scale, equipment, etc. Thus, the dropping time is preferably about 5 minutes to 2 hours, more preferably 10 minutes to 1 hour. When an alkali metal carbonate or/and an alkali metal hydrogen carbonate are used as bases, carbonic acid gas will be evolved, and thus in the case of, for example, a reaction vessel or situation from which carbonic acid gas is less easily discharged, the dropping time is still more preferably 20 minutes to 40 minutes, particularly preferably about 30 minutes, from the viewpoint of the reaction rate.

### (Alkali Metal Iodide)

In the etherification reaction in the step (i) of the production method according to the present invention, the reaction is preferably carried out in the presence of an alkali metal iodide. Specific examples of the alkali metal iodide include potassium iodide, sodium iodide, cesium iodide, and lithium iodide. These may be used alone or as a mixture of two or more. Of these, potassium iodide and sodium iodide are preferred.

The amount of the alkali metal iodide used is preferably in the range of 1 to 25 mol %, more preferably in the range of 2 to 15 mol %, still more preferably in the range of 2.5 to 10 mol %, particularly preferably in the range of 3 to 5 mol %, relative to the amount of the 1,1′-binaphthyl-2,2′-diol compound represented by formula (1).

<Step (ii)>

The step (ii) in the production method according to the present invention is a step of hydrolyzing the 2,2′-bis(alkoxycarbonylmethoxy)-1,1′-binaphthyl compound represented by formula (4) obtained through the step (i) and then performing an acid treatment. As the 2,2′-bis(alkoxycarbonylmethoxy)-1,1′-binaphthyl compound represented by formula (4) for use in the hydrolysis reaction in the step (ii), the reaction liquid obtained by the etherification reaction in the step (i) may be used without any treatment; alternatively, an oil layer obtained by performing a water-washing operation on the reaction liquid may be used, or a solid or crystal taken out from the oil layer or the reaction liquid by crystallization or the like may be used.

### (Hydrolysis)

In the hydrolysis of the 2,2′-bis(alkoxycarbonylmethoxy)-1,1′-binaphthyl compound represented by formula (4), it is preferable to use a base. Specific examples of the base for use include alkali metal hydroxides such as lithium hydroxide, sodium hydroxide, and potassium hydroxide, and by using such a base, an alkali metal salt of the 2,2′-bis(carboxymethoxy)-1,1′-binaphthyl compound represented by formula (2) can be obtained. The base used may be added to a reaction vessel as a solid or in the form of an aqueous solution. The concentration in the case where the base is used in the form of an aqueous solution is preferably 10 to 60 wt %, more preferably 20 to 50 wt %. The amount of such a base used is preferably 2.0 to 6.0 mol, more preferably 2.5 to 4.0 mol, relative to 1 mol of the 2,2′-bis(alkoxycarbonylmethoxy)-1,1′-binaphthyl compound represented by formula (4).

The amount of water used for the hydrolysis, including water contained in the aqueous solution of the base to be added, is preferably 10 to 200 parts by weight, more preferably 20 to 150 parts by weight, relative to 100 parts by weight of the 2,2′-bis(alkoxycarbonylmethoxy)-1,1′-binaphthyl compound represented by formula (4).

The reaction temperature is typically in the range of 30 °C to 100 °C, preferably in the range of 50 °C to 90 °C, more preferably in the range of 60 °C to 80 °C, and it is preferable to add or drop the base or the aqueous solution thereof while maintaining this temperature.

In the hydrolysis, a reaction solvent need not be used, but is preferably used for reasons of, for example, ease of operation in industrial production and improvement in reaction rate. The reaction solvent is not particularly limited as long as it does not distill out of a reaction vessel at a reaction temperature and is inert to the reaction, and examples include chain or cyclic ketone solvents having 5 to 8 carbon atoms, such as diethyl ketone, methyl isobutyl ketone, methyl amyl ketone, 2-octanone, cyclopentanone, and cyclohexanone, and chain nitrile solvents having 2 to 6 carbon atoms, such as acetonitrile and propanenitrile. The amount of the solvent used is preferably 100 to 600 parts by weight, more preferably 130 to 400 parts by weight, relative to 100 parts by weight of the 2,2′-bis(alkoxycarbonylmethoxy)-1,1′-binaphthyl compound of formula (4).

### (Acid Treatment)

To obtain a 2,2′-bis(carboxymethoxy)-1,1′-binaphthyl compound by performing an acid treatment on the alkali metal salt of the 2,2′-bis(carboxymethoxy)-1,1′-binaphthyl compound represented by formula (2) produced by the hydrolysis reaction, an acid may be added to the reaction liquid containing the alkali metal salt, or the alkali metal salt may be collected as a solid by crystallization, dissolved in a solvent, and subjected to the acid treatment. When the reaction liquid is used, the produced alcohol may be removed by distillation as needed.

Specific examples of the acid for use include inorganic acids such as hydrogen chloride, hydrogen bromide, and sulfuric acid, and organic acids such as p-toluenesulfonic acid, methanesulfonic acid, and trifluoromethanesulfonic acid. Hydrogen chloride is preferably used in the form of concentrated hydrochloric acid. The amount of such an acid used is preferably 2.2 to 4.0 mol, more preferably 2.5 to 3.0 mol, relative to 1 mol of the 2,2′-bis(alkoxycarbonylmethoxy)-1,1′-binaphthyl compound represented by formula (4), which is a reactant of the above hydrolysis reaction.

In the case where the reaction liquid obtained by the hydrolysis reaction is used, when the base is used in excess and left behind such that the number of moles of the unreacted base is larger than the number of moles of the alkali metal salt, this is preferably neutralized before the above amount of the acid is used.

The solvent in the acid treatment is preferably a chain or cyclic ketone solvent, more preferably a chain or cyclic ketone solvent having 5 to 8 carbon atoms, still more preferably a chain ketone solvent having 5 to 8 carbon atoms. Specific examples include diethyl ketone, methyl isobutyl ketone, methyl amyl ketone, 2-octanone, cyclopentanone, and cyclohexanone. The amount of the solvent used is preferably 250 to 1050 parts by weight, more preferably 350 to 900 parts by weight, relative to 100 parts by weight of the 2,2′-bis(alkoxycarbonylmethoxy)-1,1′-binaphthyl compound represented by formula (4), which is a reactant of the above hydrolysis reaction, or 100 parts by weight of the alkali metal salt of the 2,2′-bis(carboxymethoxy)-1,1′-binaphthyl compound represented by formula (2).

The temperature of the acid treatment is typically in the range of 30 °C to 100 °C, preferably in the range of 50 °C to 90 °C, more preferably in the range of 75 °C to 85 °C.

After the acid treatment, if necessary, a water-washing operation involving addition of water or a solvent separable from water, separation of an aqueous layer, followed by addition of water to the resulting oil layer, stirring followed by standing, and removal of an aqueous layer is preferably performed once or a plurality of times to reduce or remove the produced salt and acid from the oil layer.

<Step (iii)>

### (Crystallization)

The step (iii) in the production method according to the present invention is a step of crystallizing the 2,2′-bis(carboxymethoxy)-1,1′-binaphthyl compound represented by formula (2) obtained through the step (ii). An oil layer containing the 2,2′-bis(carboxymethoxy)-1,1′-binaphthyl compound represented by formula (2) obtained through the step (ii) may be crystallized to thereby precipitate the 2,2′-bis(carboxymethoxy)-1,1′-binaphthyl compound, or the crystallization may be performed with a solvent added and dissolved in a residual liquid obtained by solvent removal from the oil layer by, for example, distillation or with the solvent in the oil layer replaced with another solvent.

The solvent used for the crystallization may be, for example, a chain or cyclic ketone solvent, a cyclic ether solvent, or a cyclic ester solvent, and is preferably a chain or cyclic ketone solvent having 5 to 8 carbon atoms or a cyclic ether or cyclic ester having 4 to 8 carbon atoms, more preferably a chain ketone solvent having 5 to 8 carbon atoms. Specific examples include diethyl ketone, methyl isobutyl ketone, methyl amyl ketone, 2-octanone, cyclopentanone, cyclohexanone, tetrahydrofuran, 1,4-dioxane, γ-butyrolactone, and γ-valerolactone. In the case of a chain ketone having 5 to 8 carbon atoms, the amount of the solvent used is preferably 250 to 1000 parts by weight, more preferably 300 to 800 parts by weight, still more preferably 400 to 600 parts by weight, relative to 100 weights of the 2,2′-bis(carboxymethoxy)-1,1′-binaphthyl compound represented by formula (2) used in the step (iii), and when the crystallization solvent is a cyclic ketone solvent, a cyclic ether solvent, or a cyclic ester solvent, the amount thereof used is preferably 50 to 600 parts by weight, more preferably 50 to 400 parts by weight, still more preferably 100 to 200 parts by weight, relative to 100 parts by weight of the 2,2′-bis(carboxymethoxy)-1,1′-binaphthyl compound represented by formula (2) used in the step (iii).

A crystal of the 2,2′-bis(carboxymethoxy)-1,1′-binaphthyl compound represented by formula (2) can be precipitated by a known method, for example, by preparing a solution of the 2,2′-bis(carboxymethoxy)-1,1′-binaphthyl compound represented by formula (2) in the above solvent or the like and cooling the solution or distilling out the solvent.

By performing crystallization under the above conditions and separating the precipitated crystal by filtration, a target having a good hue can be obtained. From the viewpoint of obtaining a target having a good hue, it is more preferable to further crystallize the obtained target, that is, to perform the crystallization in the step (iii) twice.

### (Drying)

The crystal obtained by the crystallization in the step (iii) is preferably dried. The drying method is not particularly limited, and the crystal may be air-dried at room temperature. To prevent decreases in purity and hue, the crystal is preferably dried under reduced pressure or/and in an inert gas such as nitrogen. Industrially, the drying is preferably performed at 70 °C to 110 °C.

In the production method according to the present invention, all of the steps (ii) to (iii) are preferably performed under an atmosphere or stream with an oxygen concentration of 6 vol % or less. The oxygen concentration is preferably 5 vol % or less, more preferably 4.5 vol % or less, still more preferably 4 vol % or less. Performing the steps (ii) to (iii) under an atmosphere or stream of an inert gas such as nitrogen, argon, or helium is particularly preferred to obtain a 2,2′-bis(carboxymethoxy)-1,1′-binaphthyl compound represented by formula (2) having a good hue.

### EXAMPLES

The present invention will now be described more specifically with reference to Examples, but it should be noted that the present invention is not limited to these Examples.

Analysis methods are as follows.

### <Analysis Methods>

### 1. Determination of Purity

Measurement apparatus: high-performance liquid chromatography analyzer (manufactured by Shimadzu Corporation)
Pump: LC-20AD
Column oven: CTO-20A
Detector: SPD-20A
Column: HALO-C18
Oven temperature: 50 °C.
Flow rate: 0.7 mL/min
Detection wavelength: 280 nm
Mobile phase: (A) acetonitrile, (B) 0.1 vol % aqueous phosphoric acid solution
Gradient conditions: (A) vol % (time from start of analysis)
30% (0 min)→100% (12 min)→100% (15 min)

### 2. Hue (APHA)

A crystal was dissolved in tetrahydrofuran (manufactured by FUJIFILM Wako Pure Chemical Corporation, purity 97% or more) to obtain a 30 wt % solution, and after “standard calibration” of the following measuring instrument was performed using tetrahydrofuran, the melt color of the 30 wt % solution was measured.
Measuring instrument: TZ6000 (manufactured by Nippon Denshoku Industries Co., Ltd.)

### Example 1

A four-necked flask was charged with 60 g of 1,1′-binaphthalene-2,2′-diol, 150 g of methyl isobutyl ketone, 60.8 g of potassium carbonate, and 1.2 g of potassium iodide and purged with nitrogen, and the oxygen concentration was confirmed to be 0 vol % with an oxymeter. The temperature was raised to 100 °C, and 60 g of methyl isobutyl ketone was distilled out under reduced pressure, after which repressurization was performed with nitrogen, and the oxygen concentration was confirmed to be 0 vol %. A mixed solution of 56.9 g of ethyl chloroacetate and 0.5 g of N-methylpyrrolidone was prepared, and then the mixed solution was added dropwise while the temperature of the reaction liquid was maintained at 90 °C to 100 °C. After stirring for 10 hours, the oxygen concentration was confirmed to be 0 vol %, and the temperature was raised to 80 °C with 240 g of water added, after which the resulting mixture was allowed to stand, and an aqueous layer was removed (step i). The subsequent operations were also performed in the nitrogen gas atmosphere.

To the obtained oil layer, 180 g of methyl isobutyl ketone was added, and then 52.3 g of a 48% aqueous sodium hydroxide solution was added dropwise while the temperature of the reaction liquid was maintained at 80 °C to 85 °C. After stirring for 2 hours, 240 g of methyl isobutyl ketone was added to the reaction liquid, and 240 g of methyl isobutyl ketone, ethanol, and water was distilled out under reduced pressure while the temperature of the reaction liquid was maintained at 70 °C to 85 °C. After 180 g of water and 318 g of methyl isobutyl ketone were added, 65.4 g of concentrated hydrochloric acid was added dropwise while the temperature was maintained at 80 °C to 85 °C, and stirring was performed at the same temperature for 30 minutes. After standing, an aqueous layer was extracted, and a water-washing operation involving addition of water to the obtained oil layer, stirring, and removal of an aqueous layer by separation was performed twice (step ii).

Subsequently, under normal pressure, 390 g of water and methyl isobutyl ketone was distilled out of the obtained oil layer by distillation. At 95 °C midway through the distillation, a seed crystal obtained by a conventionally known production method was added, and precipitation of a crystal was checked. The resulting product was cooled to 30 °C at a cooling rate of 10 °C per hour and filtered to obtain 75 g of a 2,2′-bis(carboxymethoxy)-1,1′-binaphthyl with APHA 60 (step iii).

### (Recrystallization of Target)

A four-necked flask was charged with 60 g of the crystal obtained through the step (iii), 350 g of methyl isobutyl ketone, and 86 g of water and purged with nitrogen, and the oxygen concentration was confirmed to be 0% with an oxymeter. (The subsequent operations were also performed in the nitrogen gas atmosphere.) Thereafter, the mixture was heated to 85 °C and stirred to dissolve. After standing, an aqueous layer was extracted, and a water-washing operation involving addition of water to the obtained oil layer, stirring, and removal of an aqueous layer by separation was performed once. Subsequently, under normal pressure, 200 g of water and methyl isobutyl ketone was distilled out by distillation. At 95 °C midway through the distillation, a seed crystal obtained by a conventionally known production method was added, and precipitation of a crystal was checked. Thereafter, the crystallization liquid was cooled to 25 °C at a cooling rate of 10 °C per hour, filtered, and then dried under reduced pressure to obtain 56.0 g of a 2,2′-bis(carboxymethoxy)-1,1′-binaphthyl with purity 99.9% and APAH 30.

### Example 2

A four-necked flask was charged with 60 g of 1,1′-binaphthalene-2,2′-diol, 150 g of methyl isobutyl ketone, 60.8 g of potassium carbonate, and 1.2 g of potassium iodide and purged with a gas mixture of air and nitrogen with an oxygen concentration of 3.5%, and the oxygen concentration was confirmed to be 4 vols with an oxymeter. The temperature was raised to 100 °C, and 67 g of methyl isobutyl ketone was distilled out under reduced pressure, after which repressurization was performed with the gas mixture, and the oxygen concentration was confirmed to be 4 vol %. A mixed solution of 56.9 g of ethyl chloroacetate and 0.5 g of N-methylpyrrolidone was prepared, and then the mixed solution was added dropwise while the temperature of the reaction liquid was maintained at 90 °C to 100 °C. After stirring for 10 hours, the oxygen concentration was confirmed to be 4%, after which nitrogen purging was performed, and the oxygen concentration was confirmed to be 0%. The subsequent operations were performed in the nitrogen gas atmosphere. To the reaction liquid, 240 g of water was added, and the temperature was raised to 80 °C, after which the resulting mixture was allowed to stand, and an aqueous layer was removed (step i).

To the obtained oil layer, 182 g of methyl isobutyl ketone was added, and then 52.4 g of a 48% aqueous sodium hydroxide solution was added dropwise while the temperature of the reaction liquid was maintained at 80 °C to 85 °C. After stirring for 2 hours, 242 g of methyl isobutyl ketone was added to the reaction liquid, and 241 g of methyl isobutyl ketone, ethanol, and water was distilled out under reduced pressure while the temperature of the reaction liquid was maintained at 70 °C to 85 °C. After 180 g of water and 318 g of methyl isobutyl ketone were added, 65.2 g of concentrated hydrochloric acid was added dropwise while the temperature was maintained at 80 °C to 85 °C, and stirring was performed at the same temperature for 30 minutes. After standing, an aqueous layer was extracted, and a water-washing operation involving addition of water to the obtained oil layer, stirring, and removal of an aqueous layer by separation was performed twice (step ii).

Subsequently, under normal pressure, 408 g of water and methyl isobutyl ketone was distilled out of the obtained oil layer by distillation. At 95 °C midway through the distillation, a seed crystal obtained by a conventionally known production method was added, and precipitation of a crystal was checked. The resulting product was cooled to 30 °C at a cooling rate of 10 °C per hour and filtered to obtain 79 g of a 2,2′-bis(carboxymethoxy)-1,1′-binaphthyl with APHA 70 (step iii).

### (Recrystallization of Target)

A four-necked flask was charged with 65 g the crystal obtained through the step (iii), 378 g of methyl isobutyl ketone, and 92 g of water and purged with nitrogen, and the oxygen concentration was confirmed to be 0% with an oxymeter. The subsequent operations were also performed in the nitrogen gas atmosphere. Thereafter, the mixture was heated to 85 °C and stirred to dissolve. After standing, an aqueous layer was extracted, and a water-washing operation involving addition of water to the obtained oil layer, stirring, and removal of an aqueous layer by separation was performed once. Subsequently, under normal pressure, 217 g of water and methyl isobutyl ketone was distilled out by distillation. At 95 °C midway through the distillation, a seed crystal obtained by a conventionally known production method was added, and precipitation of a crystal was checked. Thereafter, the crystallization liquid was cooled to 25 °C at a cooling rate of 10 °C per hour, filtered, and then dried under reduced pressure to obtain 61.7 g of a 2,2′-bis(carboxymethoxy)-1,1′-binaphthyl with purity 99.9% and APHA 30.

### Comparative Example 1

A four-necked flask was charged with 60 g of 1,1′-binaphthalene-2,2′-diol, 150 g of methyl isobutyl ketone, 61.0 g of potassium carbonate, and 1.2 g of potassium iodide and purged with instrument air. The temperature was raised to 100 °C, and 63 g of methyl isobutyl ketone was distilled out under reduced pressure, after which repressurization was performed with air. A mixed solution of 56.9 g of ethyl chloroacetate and 0.5 g of N-methylpyrrolidone was prepared, and then the mixed solution was added dropwise while the temperature of the reaction liquid was maintained at 90 °C to 100 °C. After stirring for 10 hours, nitrogen purging was performed, and the oxygen concentration was confirmed to be 0%. The subsequent operations were performed in the nitrogen gas atmosphere. After the temperature was raised to 80 °C with 240 g of water added, the resulting mixture was allowed to stand, and an aqueous layer was removed.

To the obtained oil layer, 181 g of methyl isobutyl ketone was added, and then 54.2 g of a 48% aqueous sodium hydroxide solution was added dropwise while the temperature of the reaction liquid was maintained at 80 °C to 85 °C. After stirring for 2 hours, 240 g of methyl isobutyl ketone was added to the reaction liquid, and 244 g of methyl isobutyl ketone, ethanol, and water was distilled out under reduced pressure while the temperature of the reaction liquid was maintained at 70 °C to 85 °C After 180 g of water and 319 g of methyl isobutyl ketone were added, 69.5 g of concentrated hydrochloric acid was added dropwise while the temperature was maintained at 80 °C to 85 °C, and stirring was performed at the same temperature for 30 minutes. After standing, an aqueous layer was extracted, and a water-washing operation involving addition of water to the obtained oil layer, stirring, and removal of an aqueous layer by separation was performed twice.

Subsequently, under normal pressure, 400 g of water and methyl isobutyl ketone was distilled out of the obtained oil layer by distillation. At 95 °C midway through the distillation, a seed crystal obtained by a conventionally known production method was added, and precipitation of a crystal was checked. The resulting product was cooled to 30 °C at a cooling rate of 10 °C per hour and filtered to obtain 77 g of a 2,2′-bis(carboxymethoxy)-1,1′-binaphthyl with APHA 370.

### (Recrystallization of Target)

A four-necked flask was charged with 60 g of the obtained crystal of target A, 356 g of methyl isobutyl ketone, and 86 g of water and purged with nitrogen, and the oxygen concentration was confirmed to be 0% with an oxymeter. The subsequent operations were also performed in the nitrogen gas atmosphere. Thereafter, the mixture was heated to 85 °C and stirred to dissolve. After standing, an aqueous layer was extracted, and a water-washing operation involving addition of water to the obtained oil layer, stirring, and removal of an aqueous layer by separation was performed once. Subsequently, under normal pressure, 210 g of water and methyl isobutyl ketone was distilled out by distillation. At 95 °C midway through the distillation, a seed crystal obtained by a conventionally known production method was added, and precipitation of a crystal was checked. Thereafter, the crystallization liquid was cooled to 25 °C at a cooling rate of 10 °C per hour, filtered, and then dried under reduced pressure to obtain 57.6 g of a 2,2′-bis(carboxymethoxy)-1,1′-binaphthyl with purity 99.9% and APHA 100.

1. A method for producing a 2,2′-bis(carboxymethoxy)-1,1′-binaphthyl compound represented by formula (2), comprising performing steps (i) to (iii) below in sequence using a 1,1′-binaphthyl-2,2′-diol compound represented by formula (1) as a starting material:
(i): a step of subjecting the 1,1′-binaphthyl-2,2′-diol compound represented by formula (1) and a halogenated acetic acid ester compound represented by formula (3) to an etherification reaction under an atmosphere or stream with an oxygen concentration of 6 vol % or less to produce a 2,2′-bis(alkoxycarbonylmethoxy)-1,1′-binaphthyl compound represented by formula (4),
(ii): a step of hydrolyzing the 2,2′-bis(alkoxycarbonylmethoxy)-1,1′-binaphthyl compound represented by formula (4) and then performing an acid treatment, and
(iii): a step of performing crystallization to obtain the 2,2′-bis(carboxymethoxy)-1,1′-binaphthyl compound represented by formula (2): 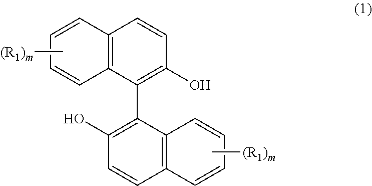
wherein each R1 independently represents a linear or branched alkyl group having 1 to 10 carbon atoms, a cyclic alkyl group having 5 to 10 carbon atoms, or an aromatic hydrocarbon group having 6 to 10 carbon atoms, and each m independently represents 0 or an integer of 1 to 4, 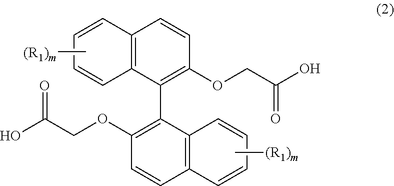
wherein R1 and m are as defined in formula (1), 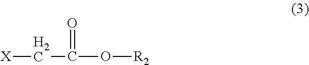
wherein X represents a halogen atom, and R2 represents a linear or branched alkyl group having 1 to 10 carbon atoms, 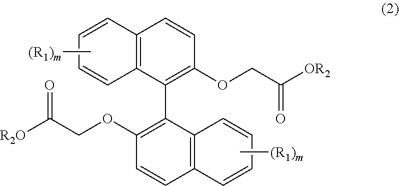
wherein R1 and m are as defined in formula (1), and each R2 is independently as defined in formula (3).

2. The method for producing a 2,2′-bis(carboxymethoxy)-1,1′-binaphthyl compound according to claim 1, wherein the crystallization in the step (iii) is performed twice.

3. The method for producing a 2,2′-bis(carboxymethoxy)-1,1′-binaphthyl compound according to claim 1, wherein all of the steps (ii) to (iii) are performed under an atmosphere or stream of an inert gas.
